(19) United States
(12) Patent Application Publication
(10) Pub. No.: US 2025/0213880 A1
(43) Pub. Date: Jul. 3, 2025

(54) MAGNETIC FIELD TREATMENT DEVICE PROVIDING MULTIPLE STIMULATION PATTERNS

(71) Applicant: MR INC., Ansan-si, Gyeonggi-do (KR)

(72) Inventors: Kyoung Sug SONG, Ansan-si, Gyeonggi-do (KR); Se Un KIM, Ansan-si, Gyeonggi-do (KR)

(21) Appl. No.: 18/852,476

(22) PCT Filed: Apr. 13, 2022
(86) PCT No.: PCT/KR2022/005320

§ 371 Date: Sep. 29, 2024

(30) Foreign Application Priority Data
Apr. 8, 2022 (KR) 10-2022-0043965

(51) Int. Cl. A61N 2/02; A61N 2/00

(52) U.S. Cl. CPC A61N 2/02; A61N 2/006

(57) ABSTRACT

The present disclosure relates to a magnetic field treatment device for providing multiple stimulation patterns, wherein the magnetic field treatment device irradiates the human body with a pulsed electromagnetic field for the purpose of pain treatment, paralysis treatment, nerve stimulation, strengthening muscles, or the like. According to an aspect of the present invention, the magnetic field treatment device comprises: a plurality of magnetic field generation circuits; and a single magnetic field discharge unit that includes a coil unit assembly composed of a plurality of coil units. The magnetic field generation circuits are respectively connected to the plurality of coil units of the coil unit assemblies, and the coil unit assembly is configured to output at least two or more output stimulation patterns.

### TECHNICAL FIELD

The present disclosure relates to a magnetic field treatment device and, more specifically, to a magnetic field treatment device for providing multiple stimulation patterns that irradiates the human body with a pulsed magnetic field for the purpose of pain treatment, paralysis treatment, nerve stimulation, strengthening muscles strengthening, or the like.

### BACKGROUND ART

In general, a magnetic field treatment device (or a stimulation treatment device) has been known as a device that is used for treatment of body parts using a magnetic field and is a device using the principle that a magnetic field is induced by applying a current to a coil and the magnetic field generated at the coil acts inside a body to generate a bioelectric current, which brings electrophysiological effects in each tissue of the human body.

In general, the magnetic field treatment devices are composed of a power supply that generates a pulsed current, a controller for controlling the power supply, and an electromagnetic coil that generates a magnetic field using the generated pulsed current. The frequency and intensity of a pulse current that is applied to a coil are adjusted to reflect the use conditions of the treatment devices in accordance with the kinds of diseases or the locations of affected areas. When a pulse current of a single frequency is used, patients are quickly adapted to stimulation; but in this case, treatment using a magnetic field is not effectively continued, so the treatment time is increased.

When stimulation of the same pattern is continuously applied to the nerves or muscles of a human body as described above, there is a tendency to become desensitized to stimulation through adaptation. Further, even when there is a problem with nerves or the function of peripheral sensory nerves is impaired due to diseases, etc., users become more desensitized to continuous stimulation than normal people, so they require stronger stimulation, but strong stimulation may lead to muscle fatigue or tissue damage.

In order to solve this problem, existing stimulators using an electromagnetic field solve such sensory problem (desensitization) by periodically changing the output intensity or the output frequency, but they also repeat stimulation of similar patterns, so the effect is negligible. Further, when the frequency of a pulse current is changed, patients may feel stimulation pain, and when the frequency is greatly changed, the stimulation pain that patients feel increases, which becomes a source of dissatisfaction.

Further, it is required to a greater pulsed current through a power supply in order to generate stronger stimulation, which requires a power supply with high output specifications, making it difficult to reduce the size of stimulation treatment devices and the cost of the devices.

### CITATION LIST

### Patent Literature

### [Patent Literature 1]

Korean Patent Application Publication No. 10-2009-0063618 (Dec. 14, 2007)

### SUMMARY OF INVENTION

### Technical Problem

The present disclosure has been made in an effort to solve the problems described above, and an objective of the present disclosure is to provide a magnetic field treatment device that simultaneously generates two or more stimulation patterns in a single electromagnetic coil, and thus can efficiently stimulate nerves and muscles.

Another objective of the present disclosure is to provide a magnetic field treatment device that can generate and supply intensive stimulation using a power supply with low output and can enable to reduce the size and manufacturing costs of the magnetic field treatment devices.

### Solution to Problem

In order to achieve the objectives described above, according to an aspect of the present disclosure, a magnetic field treatment device providing multiple stimulation patterns is provided. The magnetic field treatment device includes: a plurality of magnetic field generation circuits; and a single or a plurality of magnetic field discharge units that includes a coil unit assembly composed of a plurality of coil units, wherein the magnetic field generation circuits are respectively connected to the plurality of coil units of the coil unit assembly, and the coil unit assembly is configured to output at least two or more output stimulation patterns.

In the aspect described above, the magnetic field generation circuits each comprises a power unit providing power for generating a magnetic field, a large-capacity capacitor for accumulating power from the power unit, and a switching unit for charging and discharging the large-capacity capacitor.

In any one aspect described above, the magnetic field treatment device further includes a control unit connected to the plurality of magnetic field generation circuits, and the control unit is connected to the power unit and the switching unit.

Further, in any one aspect described above, the control unit is configured to control a magnitude and a frequency of pulse power that is supplied to each of the plurality of coil units.

Further, in any one aspect described above, the single magnetic field discharge unit simultaneously generates output stimulation patterns having multiple frequencies and multiple intensities from a single coil unit assembly, depending on the number of coil units and combinations of stimulation patterns.

Further, in any one aspect described above, output intensity of the output stimulation patterns that are generated by the single coil unit assembly is increased by magnetic fields overlapping at a joint of the plurality of coil units.

### Advantageous Effects of Invention

According to the present disclosure, it is possible to provide a magnetic field treatment device that simultaneously generates two or more stimulation patterns in a single electromagnetic coil, thus can efficiently stimulate nerves and muscles. When two or more stimulation pattern is simultaneously generated, it is possible to solve the problem of desensitization to stimulation and also possible to provide an improved treatment effect through effects such as muscle and nerve stimulation, peripheral blood circulation improvement, tissue healing, inflammation relief, or the like.

Further, according to the present disclosure, it is possible to provide a magnetic field treatment device that can generate and supply more intensive stimulation using a power supply with low output and that can reduce the size and manufacturing costs of magnetic field treatment devices.

### BRIEF DESCRIPTION OF DRAWINGS

FIG. 1 is a view schematically showing the external appearance of a magnetic field treatment device providing multiple stimulation patterns according to the present disclosure.

FIG. 2 is a block diagram schematically showing the internal structure of the magnetic field treatment device according to the present disclosure.

FIG. 3 is a view showing a double coil assembly composed of a double coil that is used in the magnetic field treatment device according to the present disclosure.

FIG. 4 is a view showing an example of an output stimulation pattern from the double coil assembly shown in FIG. 3.

FIG. 5 is a view showing another example of an output stimulation pattern from the double coil assembly shown in FIG. 3.

FIG. 6 is a view showing an example of an output stimulation pattern from a triple coil assembly.

FIG. 7 is a view showing a magnetic field distribution profile at a single coil and a multiple coil.

FIG. 8 is a view showing a modified example of a double coil assembly composed of a multiple coil that is used in the magnetic field treatment device according to the present disclosure.

### DESCRIPTION OF EMBODIMENTS

Specific structural and functional description regarding embodiments according to the concept of the present disclosure disclosed herein is exemplified only to describe the embodiments according to the concept of the present disclosure, and the embodiments according to the concept of the present disclosure may be implemented in various ways and are not limited to the embodiments described herein.

Embodiments described herein may be changed in various ways and may have various forms, so specific embodiments are shown in the drawings and will be described in detail in this specification. However, it should be understood that the exemplary embodiments according to the concept of the present disclosure are not limited to the specific examples, but modifications, equivalents, and substitutions are included in the scope and spirit of the present disclosure. Hereafter, embodiments are described in detail with reference to the accompanying drawings. However, the scope of the present disclosure is not limited to the embodiments.

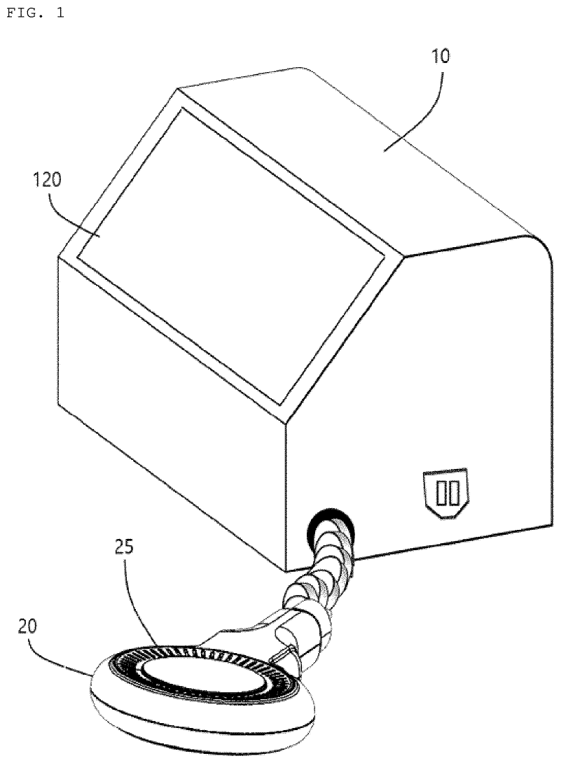

FIG. 1 is a view schematically showing an example of a magnetic field treatment device providing multiple stimulation patterns according to the present disclosure. As shown in FIG. 1, a magnetic field treatment device according to the present disclosure includes a body 10 and a magnetic field discharge unit 20. Electrical elements for generating a magnetic field, such as a power unit, a large-capacity capacitor, and a switching unit, are installed in the body 10, and a control panel 120 for a user to control the magnetic field treatment device is provided on the front of the body 10.

A user can adjust electrical elements such as the intensity, frequency, and time of pulse power related to distribution of the magnetic field output from the magnetic field discharge unit 20 through the control panel 120, and the control panel 120 may be configured to adjust magnetic field elements through physical buttons or, if a touch panel is provided, the control panel 120 may be configured to adjust magnetic field elements through a graphic user interface (GUI) provided in the touch panel screen.

The magnetic field discharge unit 20 has a magnetic field generation coil therein to provide magnetic field stimulation, which corresponds to the intensity, frequency, and time of pulse power determined by a user through the control panel 120, to the surface of the user's body. The magnetic field generation coil may include at least two or more coil units, which will be described below.

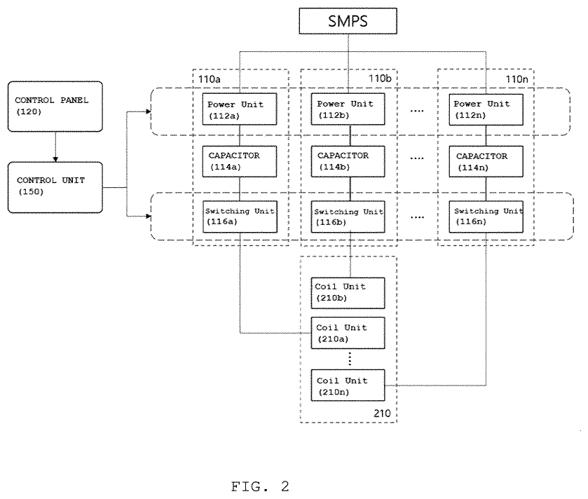

FIG. 2 is a functional block diagram functionally showing the internal structure of the magnetic field treatment device shown in FIG. 1. As shown in FIG. 2, the magnetic field treatment device includes a plurality of magnetic field generation circuits 110, the magnetic field generation circuits 110 each include a power unit 112, a large-capacity capacitor 114, and a switching unit 116, and each of the magnetic field generation circuits 110 is connected to magnetic field generation coils (coil layer). That is, the magnetic field generation circuits 110a to 110n supply pulse voltage to the magnetic field generation coils connected thereto, respectively, whereby an eddy current flows through the coils and a magnetic field is generated around the coils, and magnetic energy obtained through this process permeates into the skin or tissues of a body.

Each of the power units 112a to 112n in the magnetic field generation circuits 110 is connected to a single switching mode power supply (SMPS), whereby they are supplied with DC power from the SMPS, increase the voltage of the supplied DC power, and then supply the DC power to large-capacity capacitors 114a to 114n connected to the rear ends thereof, respectively, the large-capacity capacitors 114a to 114n is charged with and discharge sufficient energy, and switching units 116a to 116n connected to output-side paths (coil sides), respectively, switch charging and discharging of capacitor banks 240, respectively.

It is preferable that each of the capacitors has the same capacity so that the electric charge of the power charged in the capacitors is uniformly distributed, but the present disclosure is not limited thereto and may be implemented with different capacities.

The power units 112a to 112n and the switching units 116a to 116n in the magnetic field generation circuits 110 are connected to the control panel 120 through a control unit 150. When a user sets the intensity and frequency of pulse power that is supplied to the coil through the control panel 120, the control unit 150 controls the intensity of a magnetic field by adjusting the magnitude of voltage that is output from the power units 112a to 112n and controls the switching speed of the switching units 116a to 116n, thereby being able to control an output frequency. The switching units may be various thyristors. For example, a silicon controlled rectifier (SCR), a GTO thyristor, etc. may be used.

As shown in FIG. 1 and FIG. 2, coil units 210a to 210n in the magnetic field discharge unit 20 are accommodated in a housing 25 of the single magnetic field discharge unit 20. Since each of the coil units 210a to 210n is connected to the power units 112a to 112n and the switching units 116a to 116n in each of the magnetic field generation circuits 110a to 110n, the coil units 210a to 210n each generate a magnetic field having intensity and frequency set by the control unit 150, and the magnetic field discharge unit 20 or the applicator 20 formed by combining the plurality of coil units 210a to 210n can generate multiple stimulation having different intensity and frequency.

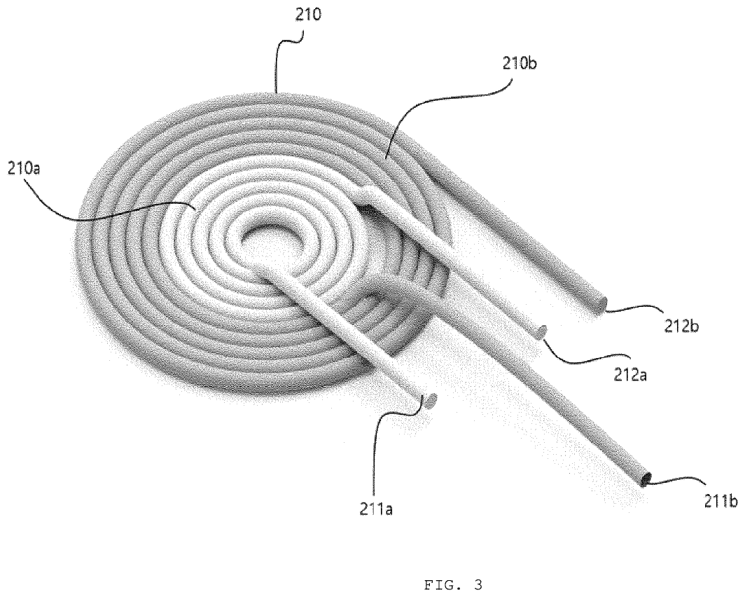

FIG. 3 is a view showing an example of a coil unit assembly 210 accommodated in the magnetic field discharge unit 20, as described above. In the embodiment shown in FIG. 3, the coil unit assembly 210 is composed of two coil units 210a and 210b, and the coil units 210a and 210b are connected to the magnetic field generation circuits 110a and 110b, respectively. For the convenience of description, the inner coil 210a of the coil unit assembly 210 is a first coil unit and connected to the first magnetic field generation circuit 110a, and the outer coil 210b of the coil unit assembly 210 is a second coil unit and connected to the second magnetic field generation circuit 110b.

Since the magnetic field discharge unit 20 is provided in a circular shape, the first coil unit 210a and the second coil unit 210b accommodated therein are also provided in circular shapes, but the present disclosure is not limited thereto and may be configured in a polygonal shape such as a triangle, a rectangle, or the like. The first coil unit 210a starts from the center inside the coil unit assembly 210 and is circumferentially wound such that the diameter increases, a first electrode terminal 211a extends from the inner end of the first coil unit and a second electrode terminal 212a extends from the outer end thereof, and each of the terminals 211a and 212a is connected to the first switching unit 116a.

Further, similarly, the second coil unit 210b starts from the inner circumference of the first coil unit 210a and is circumferentially wound such that the diameter increases, a first electrode terminal 211b extends from the inner end of the second coil unit and a second electrode terminal 212b extends from the outer end thereof, and each of the terminals 211b and 212b are connected to the second switching unit 116b.

According to this configuration, the first coil unit 210a is connected to the first power unit 112a and the first switching unit 116a in the first magnetic field generation circuit 110a, thereby generating a first magnetic field with the intensity and frequency controlled by the control unit 150. Further, the second coil unit 210b is connected to the second power unit 112b and the second switching unit 116b in the second magnetic field generation circuit 110b, thereby generating a second magnetic field with the intensity and frequency controlled by the control unit 150. Accordingly, it is possible to simultaneously generate magnetic field stimulation having different intensity and frequency through a single magnetic field discharge unit or applicator 20.

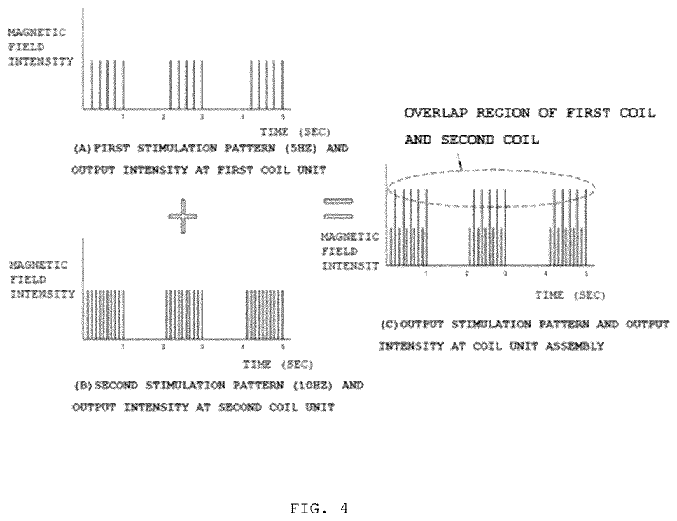

FIG. 4 is a view showing stimulation patterns and output intensity obtained by the coil unit assembly 210 shown in FIG. 3, in which (a) is a view showing a first stimulation pattern having first magnetic field intensity and a frequency of 5 Hz in the first coil unit, (b) is a view showing a second stimulation pattern having second magnetic field intensity and a frequency of 10 Hz, and (c) is a view showing a stimulation pattern in the coil unit assembly composed of the first and second coil units.

As shown in FIG. 4, the first stimulation pattern with a frequency of 5 Hz and intensity of 1 is generated in the first coil unit, and the second stimulation pattern with a frequency of 10 Hz and intensity of 1 is generated in the second coil unit. Accordingly, the stimulation pattern in the coil unit assembly composed of the first and second coil units is an output stimulation pattern having a combination of the first and second stimulation patterns, and magnetic intensities overlap each other at the interface region of the first coil and the second coil.

That is, the coil unit assembly represents high magnetic field intensity at the physical overlap position of the joint of the first coil unit at the inside and the second coil unit at the outside, and when output of the magnetic fields generated by the coil units overlap in relation to time, the output is increased by overlap at the point in time. Such an increase of output at the overlap region can generate high output intensity using low power, so it is not required to use a high-power power unit in order to provide high output intensity.

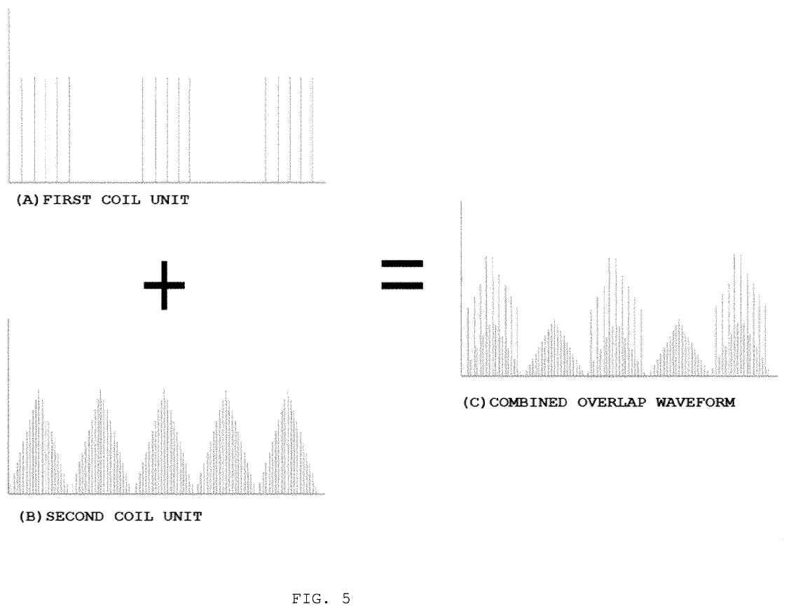

FIG. 5 is a view showing an example of other output stimulation patterns that is formed by the first coil unit and the second coil unit. As shown in FIG. 5, a first stimulation pattern with a frequency of 5 Hz and uniform output intensity is formed at the first coil unit, a second stimulation pattern with a frequency of 10 Hz and output intensity that changes over time is formed at the second coil unit, so, finally, a new stimulation pattern that is a combination of the first stimulation pattern and the second stimulation pattern is formed as shown in (c) of FIG. 5.

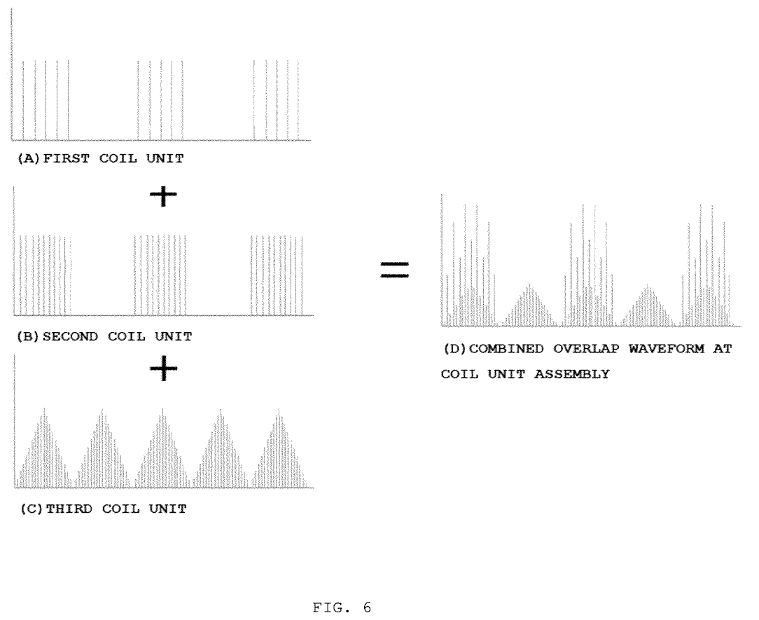

FIG. 6 is a view showing an example of output stimulation patterns that are provided when a single coil unit assembly is formed using first to third coil units. As described above, in an embodiment of the present disclosure, it is possible to implement stimulation with multiple frequencies and multiple intensities from a single coil depending on the number of coil units and combinations of stimulation patterns and possible to make various combinations of stimulation patterns by changing such stimulation patterns in relation to time, whereby it is possible to solve the problem of sensory adaptation (desensitization).

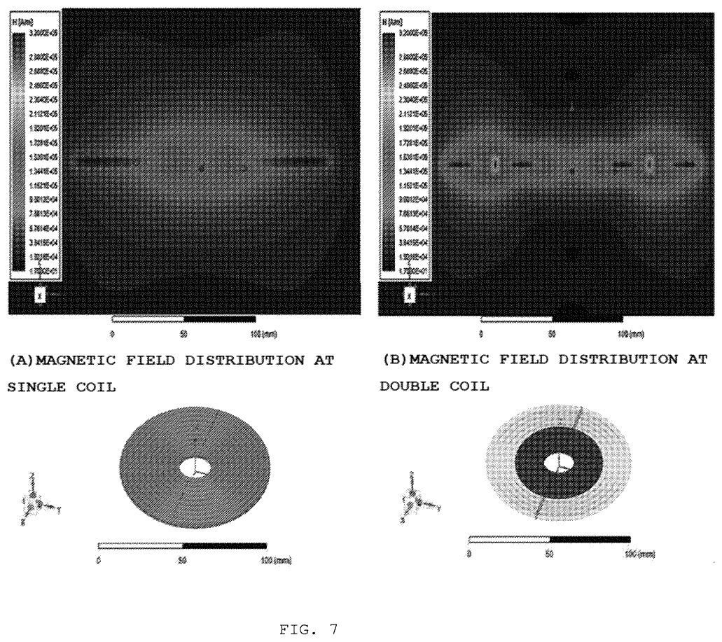

FIG. 7 is a view showing a simulation of magnetic field distribution at a single coil of the related art and magnetic field distribution at a multiple (double) coil according to an embodiment of the present disclosure. As shown in FIG. 7, a stimulation pattern is obtained in a form fixed in accordance with the intensity and frequency of a pulse current that is supplied to the coil in the single coil of the related art, but a magnetic field distribution can be obtained in a various form at the double coil according to an embodiment of the present disclosure depending on the intensity and frequency of the pulse current that is applied to the first coil and the second coil. For example, when a strong pulse current is applied to the outer coil (second coil unit) and a relatively weak pulse current is applied to the inner coil (first coil unit), as shown in FIG. 7, magnetic field distribution with a magnetic field stronger on the outside the coil unit assembly in comparison to the inside can be obtained, but, in the opposite case, that is, when a weak pulse current is applied to the outer coil (second coil unit) and a relatively strong pulse current is applied to the inner coil (first coil unit), magnetic field distribution with a magnetic field, which is opposite to that shown in (b) of FIG. 7, that is, is stronger on the inside the coil unit assembly in comparison to the outside can be obtained.

Further, in the physical region of the joint between the first coil unit and the second coil unit, magnetic fields overlap each other, so high magnetic field intensity can be obtained. The overlap magnetic field intensity can also be adjusted by adjusting the first stimulation pattern and the second stimulation pattern, whereby it is possible to provide more various stimulation patterns to skin tissues through the magnetic field assembly 210 or the magnetic field discharge unit 20.

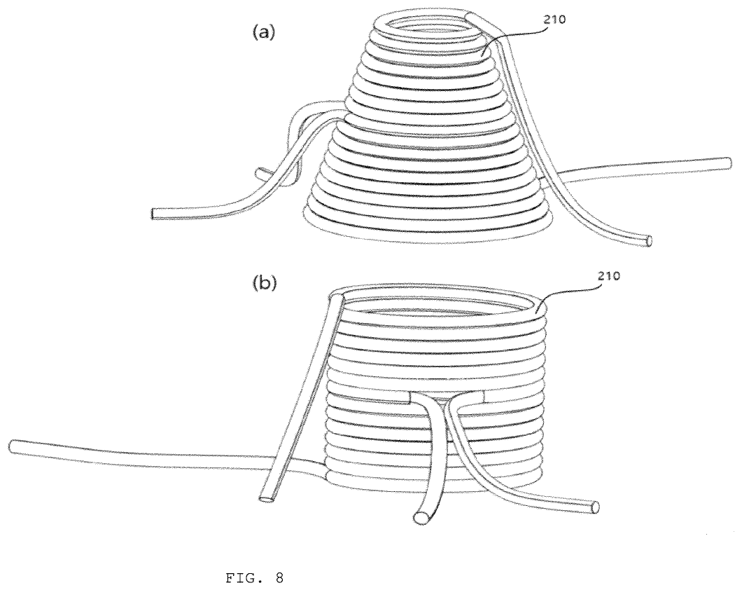

FIG. 8 is a view showing another embodiment of a coil unit assembly composed of a multiple coil according to the present disclosure. As shown in FIG. 8, a coil unit assembly of the present disclosure may be formed in a three-dimensional shape including (a) a conical coil unit assembly and (b) a cylindrical coil unit assembly, but the present disclosure is not limited thereto and may be formed in a polyhedral or polygonal cylindrical shape, and the coil unit assemblies are each composed of two or more coil unit, the same as that shown in FIG. 3, thereby providing magnetic field distribution in various forms. Such a three-dimensional coil unit assembly is applied to various body parts such as the wrist, ankle, calf, or the like of a human, thereby having an effect of being able to simultaneously provide various multiple stimulation patterns to desired parts of a body.

Embodiments were described above with reference to the limited examples and drawings, but they may be changed and modified in various ways by those skilled in the art. For example, the described technologies may be performed in orders different from the described method, and/or even if components such as the described system, structure, device, and circuit are combined or associated in different ways from the description or replaced by other components or equivalents, appropriate results can be accomplished.

Therefore, other implementations, other embodiments, and equivalents to the claims should be construed as being included in the following claims.

### REFERENCE SIGNS LIST

10: Body of magnetic field treatment device
20: Magnetic field discharge unit
25: Housing
110: Magnetic field generation circuit
112: Power unit
114: Capacitor
116: Switching unit
210: Coil unit assembly

1. A magnetic field treatment device providing multiple stimulation patterns, comprising:
a plurality of magnetic field generation circuits; and
a single or a plurality of magnetic field discharge unit that includes a coil unit assembly composed of a plurality of coil units,
wherein the magnetic field generation circuits are respectively connected to the plurality of coil units of the coil unit assembly, and the coil unit assembly is configured to output at least two or more output stimulation patterns.

2. The magnetic field treatment device of claim 1, wherein the magnetic field generation circuits each comprises a power unit providing power for generating a magnetic field, a large-capacity capacitor for accumulating power from the power unit, and a switching unit for charging and discharging the large-capacity capacitor.

3. The magnetic field treatment device of claim 2, further comprising a control unit connected to the plurality of magnetic field generation circuits,
wherein the control unit is connected to the power unit and the switching unit.

4. The magnetic field treatment device of claim 3, wherein the control unit is configured to control a magnitude and a frequency of pulse power that is supplied to each of the plurality of coil units.

5. The magnetic field treatment device of claim 1, wherein the single magnetic field discharge unit simultaneously generates output stimulation patterns having multiple frequencies and multiple intensities from a single coil unit assembly depending on the number of coil units and combinations of stimulation patterns.

6. The magnetic field treatment device of claim 5, wherein output intensity of the output stimulation patterns that are formed by the single coil unit assembly is increased by magnetic fields overlapping at a joint of the plurality of coil units.
